SEQUENCE LISTING (14 sequences)

SEQ ID NO: 1 - DNA, 64 bp, Artificial sequence
  primer
taaacatcgtcggatcggactgattacgctgcacttatacctgtgacggaagatcacttcgcag

SEQ ID NO: 2 - DNA, 63 bp, Artificial sequence
  primer
tcccggaaagtcggcccagaagaaaaggactggagcaattacgccccgccctgccactcatcg

SEQ ID NO: 3 - DNA, 935 bp, Artificial sequence
  cat gene flanked with ilvBN regions
taaacatcgtcggatcggactgattacgctgcacttatacctgtgacggaagatcacttcgcagaataaataaatcctggtgtccctgttgataccgggaagccctgggccaacttttggcgaaaatgagacgttgatcggcacgtaagaggttccaactttcaccataatgaaataagatcactaccgggcgtattttttgagttatcgagattttcaggagctaaggaagctaaaatggagaaaaaaatcactggatataccaccgttgatatatcccaatggcatcgtaaagaacattttgaggcatttcagtcagttgctcaatgtacctataaccagaccgttcagctggatattacggcctttttaaagaccgtaaagaaaaataagcacaagttttatccggcctttattcacattcttgcccgcctgatgaatgctcatccggaattccgtatggcaatgaaagacggtgagctggtgatatgggatagtgttcacccttgttacaccgttttccatgagcaaactgaaacgttttcatcgctctggagtgaataccacgacgatttccggcagtttctacacatatattcgcaagatgtggcgtgttacggtgaaaacctggcctatttccctaaagggtttattgagaatatgtttttcgtctcagccaatccctgggtgagtttcaccagttttgatttaaacgtggccaatatggacaacttcttcgcccccgttttcaccatgggcaaatattatacgcaaggcgacaaggtgctgatgccgctggcgattcaggttcatcatgccgtctgtgatggcttccatgtcggcagaatgcttaatgaattacaacagtactgcgatgagtggcagggcggggcgtaattgctccagtccttttcttctgggccgactttccggga

SEQ ID NO: 4 - DNA, 21 bp, Artificial sequence
  primer
tacgcgcttaccttaacgata

SEQ ID NO: 5 - DNA, 21 bp, Artificial sequence
  primer
ggactggaacaacacacgatt

SEQ ID NO: 6 - DNA, 1080 bp, Artificial sequence
  ilvBN::cat
tacgcgcttaccttaacgataagcgcgatgttgttcaagccttgagcggttactgaaaaaacaccgcgatcttgttaaacatcgtcggatcggactgattacgctgcactttatacctgtgacggaagatcacttcgcagaataaataaatcctggtgtccctgttgataccgggaagccctgggccaacttttggcgaaaatgagacgttgatcggcacgtaagaggttccaactttcaccataatgaaataagatcactaccgggcgtattttttgagttatcgagattttcaggagctaaggaagctaaaatggagaaaaaaatcactggatataccaccgttgatatatcccaatggcatcgtaaagaacattttgaggcatttcagtcagttgctcaatgtacctataaccagaccgttcagctggatattacggcctttttaaagaccgtaaagaaaaataagcacaagttttatccggcctttattcacattcttgcccgcctgatgaatgctcatccggaattccgtatggcaatgaaagacggtgagctggtgatatgggatagtgttcacccttgttacaccgttttccatgagcaaactgaaacgttttcatcgctctggagtgaataccacgacgatttccggcagtttctacacatatattcgcaagatgtggcgtgttacggtgaaaacctggcctatttccctaaagggtttattgagaatatgtttttcgtctcagccaatccctgggtgagtttcaccagttttgatttaaacgtggccaatatggacaacttcttcgcccccgttttcaccatgggcaaatattatacgcaaggcgacaaggtgctgatgccgctggcgattcaggttcatcatgccgtctgtgatggcttccatgtcggcagaatgcttaatgaattacaacagtactgcgatgagtggcagggcggggcgtaattgctccagtccttttcttctgggccgactttccgggaggtgcttaaacgaaaaaccccgcccggtttgcgccggcggggttttggaatcgtgtgttgttccagtcc

SEQ ID NO: 7 - DNA, 64 bp, Artificial sequence
  primer
cttttcacctttcctcctgtttattcttattaccccacaaagccacgttgtgtctcaaaatctc

SEQ ID NO: 8 - DNA, 64 bp, Artificial sequence
  PCR primer
acatgttgggctgtaaattgcgcattgagatcattccaaatccagcccttcccgatctgttgaa

SEQ ID NO: 9 - DNA, 1370 bp, Artificial sequence
  KmR gene flanked with ilvIH regions
cttttcacctttcctcctgtttattcttattaccccacaaagccacgttgtgtctcaaaatctctgatgttacattgcacaagataaaaatatatcatcatgaacaataaaactgtctgcttacataaacagtaatacaaggggtgttatgagccatattcaacgggaaacgtcttgctcgaggccgcgattaaattccaacatggatgctgatttatatgggtataaatgggctcgcgataatgtcgggcaatcaggtgcgacaatctatcgattgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgatgcatggttactcaccactgcgatccccgggaaaacagcattccaggtattagaagaatatcctgattcaggtgaaaatattgttgatgcgctggcagtgttcctgcgccggttgcattcgattcctgtttgtaattgtccttttaacagcgatcgcgtatttcgtctcgctcaggcgcaatcacgaatgaataacggtttggttgatgcgagtgattttgatgacgagcgtaatggctggcctgttgaacaagtctggaaagaaatgcataagcttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaatcagaattggttaattggttgtaacactggcagagcattacgctgacttgacgggacggcggctttgttgaataaatcgaacttttgctgagttgaaggatcagatcacgcatcttcccgacaacgcagaccgttccgtggcaaagcaaaagttcaaaatcaccaactggtccacctacaacaaagctctcatcaaccgtggctccctcactttctggctggatgatggggcgattcaggcctggtatgagtcagcaacaccttcttcacgaggcagacctcagcgctcaaagatgcaggggtaaaagctaaccgcatctttaccgacaaggcatccggcagttcaacagatcgggaagggctggatttggaatgatctcaatgcgcaatttacagcccaacatgt

SEQ ID NO: 10 - DNA, 21 bp, Artificial sequence
  primer
ttgctgtaagttgtgggattc

SEQ ID NO: 11 - DNA, 20 bp, Artificial sequence
  primer
tccaggttcccactgatttc

SEQ ID NO: 12 - DNA, 1475 bp, Artificial sequence
  PCR product
gtaagttgtgggattcagccgatttattatcaatttaatcctctgtaatggaggattttatcgtttcttttcacctttcctcctgtttattcttattaccccacaaagccacgttgtgtctcaaaatctctgatgttacattgcacaagataaaaatatatcatcatgaacaataaaactgtctgcttacataaacagtaatacaaggggtgttatgagccatattcaacgggaaacgtcttgctcgaggccgcgattaaattccaacatggatgctgatttatatgggtataaatgggctcgcgataatgtcgggcaatcaggtgcgacaatctatcgattgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgatgcatggttactcaccactgcgatccccgggaaaacagcattccaggtattagaagaatatcctgattcaggtgaaaatattgttgatgcgctggcagtgttcctgcgccggttgcattcgattcctgtttgtaattgtccttttaacagcgatcgcgtatttcgtctcgctcaggcgcaatcacgaatgaataacggtttggttgatgcgagtgattttgatgacgagcgtaatggctggcctgttgaacaagtctggaaagaaatgcataagcttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaatcagaattggttaattggttgtaacactggcagagcattacgctgacttgacgggacggcggctttgttgaataaatcgaacttttgctgagttgaaggatcagatcacgcatcttcccgacaacgcagaccgttccgtggcaaagcaaaagttcaaaatcaccaactggtccacctacaacaaagctctcatcaaccgtggctccctcactttctggctggatgatggggcgattcaggcctggtatgagtcagcaacaccttcttcacgaggcagacctcagcgctcaaagatgcaggggtaaaagctaaccgcatctttaccgacaaggcatccggcagttcaacagatcgggaagggctggatttggaatgatctcaatgcgcaatttacagcccaacatgtcacgttgggctttttttgcgaaatcagtgggaacctgga

SEQ ID NO: 13 - DNA, 28 bp, Artificial sequence
  primer
aggagctcagagttcgctgaatccttag

SEQ ID NO: 14 - DNA, 28 bp, Artificial sequence
  primer
actctagatccaggttcccactgatttc

(19) United States
(12) Patent Application Publication
(10) Pub. No.: US 2006/0057685 A1
(43) Pub. Date: Mar. 16, 2006

(54) Method for producing abnormal amino acids using a bacterium of the Enterobacteriaceae family having all acetohydroxy acid synthases inactivated

(76) Inventors: Natalia Viktorovna Stoynova, Moscow (RU); Elena Viktorovna Sycheva, Moscow Region (RU); Ekaterina Sergeevna Preobrazhenskaya, Moscow (RU); Anna Evgenievna Novikova, Moscow (RU); Nikolay Georgievich Matrosov, Moscow (RU)

Correspondence Address: CERMAK & KENEALY LLP;ACS LLC, 515 EAST BRADDOCK ROAD, SUITE B, ALEXANDRIA, VA, 22314, US

(21) Appl. No.: 11/218,787

(22) Filed: Sep. 6, 2005

(30) Foreign Application Priority Data
Sep. 10, 2004 (RU) 2004127011

(51) Int. Cl. C12P 13/04; C12N 1/21

(52) U.S. Cl. 435/106; 435/252.3

(57) ABSTRACT

A method for producing abnormal amino acids is provided, such as norleucine and norvaline, using a bacterium of Enterobacteriaceae family, particularly a bacterium belonging the genus Escherichia which has all acetohydroxy acid synthases (AHASes) inactivated.

### BACKGROUND OF THE INVENTION

1. Field of the Invention

The present invention relates to the microbiological industry, and specifically to a method for producing an abnormal amino acid, such as norleucine and norvaline, using a bacterium of Enterobacteriaceae family, wherein all acetohydroxy acid synthases (AHASes) of the bacterium are inactivated.

2. Description of the Related Art

Norleucine is known to act as methionine analog (Lawrence, J. Bacteriol., 109, 8-11 (1972)). Its ability to be incorporated into proteins instead of methionine has been demonstrated (Barker, D. and Bruton, C., J. Mol. Biol., 133, 217-31 (1979), Munier, R. and Cohen, G., Biochim. Biophys. Acta., 31, 378-90 (1959); Bogosyan, G. et al., J. Biol. Chem., 264, 1, 531-539, (1989); U.S. Pat. No. 5,622,845; U.S. Pat. No. 5,599,690). Escherichia coli growth has been reported to be prevented by competitive inhibition of methionine utilization by supplying exogenous norleucine (Harris, J. S. and Kohn, H. I., J. Pharmacol., 73, 383-400 (1941)). Norleucine supplementation has also been observed to increase the growth of an E. coli methionine auxotroph in media containing suboptimal concentrations of methionine (Lampen, J. O. and Jones, M. J., Arch. Biochem. Biophys., 13, 47-53 (1947)).

A method for producing norleucine by an E. coli strain with a high level of synthesis of leucine-rich proteins in a fermentation medium having a low concentration of amino acids has been disclosed (U.S. Pat. No. 5,622,845).

A mutant of Serratia marcescens which was depressed for the leucine biosynthetic enzymes was selected and used to further select an isoleucine-dependent mutant lacking threonine dehydratase. From this double mutant, an isoleucine-independent-mutant which contained a feedback-resistant .alpha.-isopropylmalate synthase was selected. This final complex mutant was able to cause accumulation of norleucine (Kisumi et al., J. Biochem., 1976, 80, 333-339

It has been proposed that norleucine is generated from pyruvate or 2-ketobutyrate (instead of initial substrate 2-ketoisovalerate) by the leucine biosynthesis pathway (FIG. 1) in S. marcesens (Kisumi et al., J. Biochem., 1976, 80, 333-339) and E. coli (Bogosyan, G., et al., J. Biol. Chem., 264, 1, 531-539 (1989)).

Methods for producing protein containing nonprotein amino acids by culturing a protein-producing microorganism, in particular E. coli, in the culture medium containing nonprotein amino acids, including norleucine and norvaline, has been disclosed (U.S. Pat. No. 4,879,223).

Norleucine incorporation into recombinant gene products may also be promoted by culturing the cells expressing the gene products in a media containing norleucine, or deficient in leucine and/or methionine. Thus, norleucine analogs of IL-2, G-CSF, gamma-interferon and alpha-consensus interferon may be produced in this way (U.S. Pat. No. 5,599,690). But to date, accumulation of norleucine (and norvaline) by AHAS-deficient E. coli strains has not been reported.

### SUMMARY OF THE INVENTION

An object of the present invention is to provide abnormal amino acid-producing strains. It is a further object of the present invention to provide a method for producing such abnormal amino acid using these strains.

It is a further object of the present invention to provide a bacterium of the Enterobacteriaceae family which has an ability to produce abnormal amino acids, such as norleucine and norvaline.

It is a further object of the present invention to provide an abnormal amino acid-producing bacterium of Enterobacteriaceae family, wherein the bacterium has been modified to inactivate all acetohydroxy acid synthases (AHASes) .

It is a further object of the present invention to provide the bacterium described above, wherein the abnormal amino acid is norleucine and/or norvaline.

It is a further object of the present invention to provide the bacterium as described above, wherein the bacterium belongs to the genus Escherichia.

It is a further object of the present invention to provide the bacterium described above, wherein said acetohydroxy acid synthases include AHAS I encoded by the ilvBN genes, AHAS II encoded by the ilvGM genes, and AHAS III encoded by the ilvIH genes.

It is a further object of the present invention to provide the bacterium described above, wherein the bacterium is modified to have enhanced expression of the leuABCD operon.

It is a further object of the present invention to provide a method for producing abnormal amino acids comprising:
cultivating the bacterium as described above in a medium which does not contain L-leucine, but does contain L-valine at a concentration which limits bacterial growth, so that abnormal amino acids are produced and secreted into the medium, and
collecting abnormal amino acid from the medium.

It is a further object of the present invention to provide the method described above, wherein the abnormal amino acid is norleucine and/or norvaline.

The present invention is described in detail below.

### BRIEF DESCRIPTION OF DRAWINGS

FIG. 1 shows the scheme of norleucine and norvaline biosynthesis through the keto acid chain elongation process.

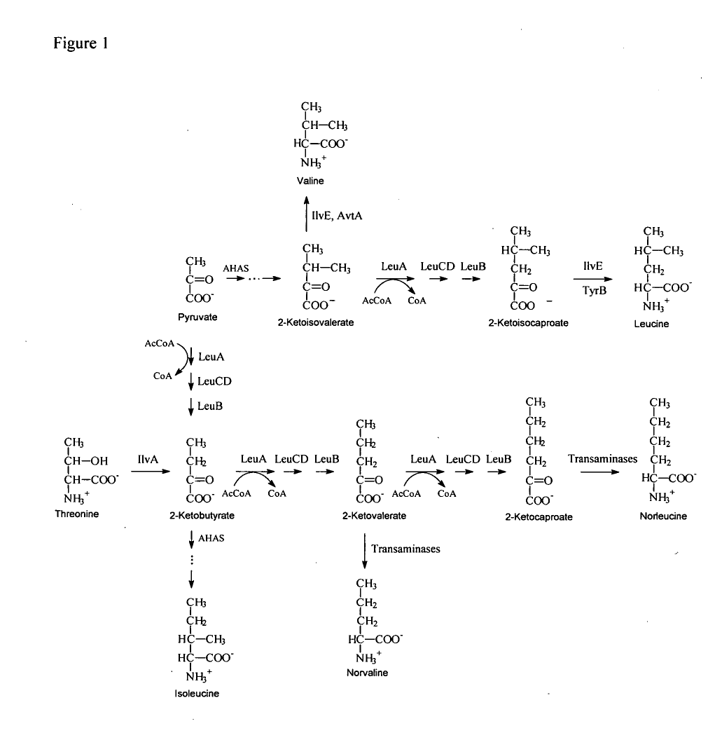

FIG. 2 shows the presumed scheme of abnormal amino acids biosynthesis in AHAS-deficient E. coli strain. KIV—2-ketoisovalerate; PYR—pyruvate; KB—2-ketobutyrate; KV—2-ketovalerate; KIC—2-ketoisocaproate; KC—2-ketocaproate; IPMS—isopropylmalate synthase; IPMI—isopropylmalate isomerase; IPMD—isopropylmalate dehydrogenase.

FIG. 3 shows the relative position of the primers ilvBN1 and ilvBN2 on the plasmid pACYC 184.

FIG. 4 shows the scheme for construction of chromosomal DNA fragment which includes the inactivated ilvBN operon.

FIG. 5 shows plasmid pMW118-ilvIH.

FIG. 6 shows plasmid pBR-leuABCD.

FIG. 7 shows HPLC separation of: a) the standard mixtures of amino acids; b) the standard mixture of amino acids containing 0.06 mM each of norvaline and norleucine; c) fermentation broth.

### DETAILED DESCRIPTION OF THE INVENTION

1. Bacterium of the Present Invention

The aforementioned objects were achieved by finding that the inactivation of all AHASes can enhance production of abnormal amino acids, such as norleucine and norvaline. Furthermore, it was shown that enhanced expression of the leuABCD operon increases production of such abnormal amino acids by AHASes-deficient strains. Thus the present invention has been completed.

The bacterium of the present invention is an abnormal amino acid-producing bacterium of Enterobacteriaceae family, wherein the bacterium has been modified to inactivate all AHASes.

In the present invention, “abnormal amino acid” means an amino acid other than the 20 basic proteinogenic amino acids (L-alanine, L-arginine, L-asparagine, L-aspartic acid, L-cysteine, L-glutamic acid, L-glutamine, L-glycine, L-histidine, L-isoleucine, L-leucine, L-lysine, L-methionine, L-phenylalanine, L-proline, L-serine, L-threonine, L-tryptophan, L-tyrosine and L-valine) and also called a “non-proteinogenic amino acid”. Such an abnormal (non-proteinogenic) amino acid can be formed from its 2-keto-precursor by enzymes of the L-leucine biosynthetic pathway encoded by the leuABCD operon. Examples of the “abnormal amino acid” according to the present invention are norvaline formed from 2-ketovalerate and norleucine formed from 2-ketovalerate through 2-ketocaproate. Other examples of the “abnormal amino acid” according to the present invention is homoleucine formed from 2-ketoisocaproate and homoisoleucine formed from 2-keto-3-methylvalerate by enzymes of L-leucine biosynthetic pathway (see, for example, Jakubowski, H. and Goldman, E., Microbiological reviews, v. 56, No. 3, p. 412-429 (1992)). Other chemical homologues of abovementioned amino acids are also encompassed by the scope of the present invention. In the present invention, the abnormal amino acid is preferably in the L-form.

In the present invention, an “abnormal amino acid-producing bacterium” means a bacterium, which has an ability to produce and secrete an abnormal amino acid into a medium, when the bacterium of the present invention is cultured in the medium. The abnormal amino acid-producing ability may be imparted or enhanced by breeding. The term “abnormal amino acid-producing bacterium” used herein also means a bacterium, which is able to produce and secrete such an abnormal amino acid into a culture medium in an amount larger than a wild-type or parental strain of E. coli, such as E. coli strains K-12, W3110 or MG 1655. Preferably this phrase means that the bacterium is able to produce and secrete into a medium an amount of not less than 10 mg/l, more preferably not less than 50 mg/l of the target amino acid.

The Enterobacteriaceae family includes, but is not limited to, bacteria belonging to the genera Escherichia, Erwinia, Providencia and Serratia. The genus Escherichia is preferred.

The phrase “a bacterium belonging to the genus Escherichia” means that the bacterium is classified as the genus Escherichia according to the classification known to a person skilled in the microbiology. An example of a microorganism belonging to the genus Escherichia as used in the present invention is Escherichia coli (E. coli).

The bacterium of the Enterobacteriaceae family that can be used in the present invention is not particularly limited, however for example, bacteria described by Neidhardt, F. C. et al. (Escherichia coli and Salmonella typhimurium, American Society for Microbiology, Washington D.C., 1208, Table 1) are encompassed.

The term “AHAS is inactivated” means that the gene coding for AHAS is modified so that the modified gene encodes a mutant protein with significantly decreased activity, particularly so activity is undetectable by conventional techniques, or such gene encodes a completely inactive protein. It is also possible that the modified DNA region is unable to naturally express the gene due to deleting part of the gene, shifting the reading frame of the gene, or modifying the adjacent regions of the operon, including sequences controlling operon expression, such as the promoter(s), enhancer(s), attenuator(s) etc.

AHASes according to the present invention include AHAS I, II and III. The phrase “all acetohydroxy acid synthases (AHASes)” typically means AHASI, II and III.

AHAS I (acetohydroxybutanoate synthase I/acetolactate synthase I), AHAS II (acetohydroxybutanoate synthase II/acetolactate synthase II) and AHAS III (acetolactate synthase III/acetohydroxybutanoate synthase III) catalyze two reactions which generate 2-aceto-2-hydroxy-butyrate from pyruvate and 2-oxobutanoate, and acetolactate from pyruvate.

AHAS I is encoded by the ilvBN genes (ilvBN operon), AHAS II is encoded by the ilvGM genes (ilvGM operon) and AHAS III is encoded by the ilvIH genes (ilvIH operon).

The ilvB gene (numbers 3850411 to 3848723 in the GenBank accession number NC_{—}000913.1) and ilvN gene (numbers 3848719 to 3848429 in the GenBank accession number NC_{—}000913.1) are located between the uhpA and ivbL genes on the chromosome of E. coli strain K-12 and encode for catalytic and regulatory subunits of AHAS I, respectively.

The ilvG gene joined numbers 3948183 to 3949163 and 3949162 to 3949827 in the GenBank accession number NC_{—}000913.1) and ilvM gene (numbers 3949824 to 3950087 in the GenBank accession number NC_{—}000913.1) are located between the ilvL and ilvE genes on the chromosome of E. coli strain K-12 and encode for large and small subunits of AHAS II, respectively.

The ilvI gene (numbers 85630 to 87354 in the GenBank accession number NC_{—}000913.1) and ilvH gene (numbers 87357 to 87848 in the GenBank accession number NC_{—}000913.1) are located between the leuO and fruR genes on the chromosome of E. coli strain K-12 and encode for large and small subunits of AHAS III, respectively.

Inactivation of the gene can be performed by conventional methods, such as mutagenesis treatment using UV irradiation or nitrosoguanidine (N-methyl-N′-nitro-N-nitrosoguanidine) treatment, site-directed mutagenesis, gene disruption using homologous recombination or/and insertion-deletion mutagenesis (Datsenko K. A. and Wanner B. L., Proc. Natl. Acad. Sci. USA, 2000, 97:12: 6640-45) also called “Red-driven integration”.

A mutant protein with significantly decreased activity or a completely inactive protein also can be obtained, for example, by site-directed mutagenesis of amino acid residues located in the conserved regions of the AHAS. Alignment of amino acid sequences of AHASes from numerous species and structure-function correlation data are presented in review of Duggleby, R. G. and Pang, S. S. (J. Biochem. Mol. Biol., 33, No. 1, 1-36 (2000)). It is stated that, for example, in AHASes from E. coli, the glutamate in 47^{th} position plays a catalytic role. So, a mutation at this position or a deletion which includes this position would be critical for enzymatic activity of AHASes from E. coli.

The ilvBN operon, ilvGM operon or ilvIH operon of a bacterium of Enterobacteriaceae family other than E. coli can also be inactivated by homologous recombination using a fragment of the ilvBN operon, ilvGM operon or ilvIH operon from E. coli or a fragment of an inherent operon which may be a homologue to the E. coli ilvBN operon, ilvGM operon or ilvIH operon. Such an operon homologue may have homology of not less than 70%, preferably not less than 80%, more preferably not less than 90%, and most preferably not less than 95% to the E. coli ilvBN operon, ilvGM operon or ilvIH operon with respect to the nucleotide sequence of the respective coding regions.

The bacterium of the present invention may be further improved by enhancing the expression of the leuABCD operon as compared with, for example, a wild-type strain or unmodified strain. The leuABCD operon includes the leuA, leuB, leuC and leuD genes. Among them, the leuA gene encodes α-isopropylmalate synthase, the leuB gene encodes β-isopropylmalate dehydrogenase, the leuC and leuD genes encode isopropylmalate isomerase.

The leuA gene (numbers 83529 to 81958 in the GenBank accession number NC_{—}000913.1), leuB gene (numbers 81958 to 80867 in the GenBank accession number NC_{—}000913.1), leuC gene (numbers 80864 to 79464 in the GenBank accession number NC_{—}000913.1) and leuD gene (numbers 79453 to 78848 in the GenBank accession number NC_{—}000913.1) are located between the leuL gene and yabM ORF on the E. coli strain K-12 chromosome.

The ilvBN operon, ilvGM operon or ilvIH operon, or leuA gene, leuB gene, leuc gene or leuD gene may include a gene encoding a protein which has the amino acid sequence described in each of the above-mentioned GenBank accession numbers and contains deletion, substitution, insertion, or addition of one or several amino acids at one or a plurality of positions, provided that the protein has an activity of AHAS, α-isopropylmalate synthase, β-isopropylmalate dehydrogenase, or isopropylmalate isomerase.

The number of “several” amino acids differs depending on the position or the type of amino acid residues in the three dimensional structure of the protein. This is for the following reason. That is, some amino acids have high homology to one another and the difference in such an amino acid does not greatly affect the three dimensional structure of the protein. Therefore, the protein of the present invention may be one which has homology of not less than 70%, preferably not less than 80%, more preferably not less than 90%, most preferably not less than 95% with respect to the entire amino acid residues.

The homolog of the ilvBN operon, ilvGM operon or ilvIH operon, or leuA gene, leuB gene, leuC gene or leuD gene of E. coli may be a DNA which hybridizes with the nucleotide sequence of the ilvBN operon, ilvGM operon or ilvIH operon, or leuA gene, leuB gene, leuC gene or leuD gene of E. coli under stringent conditions, and which codes for a protein having the corresponding enzymatic activity. The term “stringent conditions” referred to herein means conditions under which so-called specific hybrid is formed, and non-specific hybrid is not formed. It is difficult to express this condition precisely by using any numerical value. However, for example, the stringent conditions include conditions under which DNAs having high homology, for example, DNAs having homology of not less than 70%, preferably not less than 80%, more preferably not less than 90%, most preferably not less than 95% with each other are hybridized, and DNAs having homology lower than the above with each other are not hybridized.

To evaluate the degree of protein or DNA homology several calculation methods, such as a BLAST search, FASTA search and CrustalW can be used.

BLAST (Basic Local Alignment Search Tool) is the heuristic search algorithm employed by the programs blastp, blastn, blastx, megablast, tblastn, and tblastx; these programs ascribe significance to their findings using the statistical methods of Karlin, Samuel and Stephen F. Altschul (“Methods for assessing the statistical significance of molecular sequence features by using general scoring schemes”. Proc. Natl. Acad. Sci. USA, 1990, 87:2264-68; “Applications and statistics for multiple high-scoring segments in molecular sequences”. Proc. Natl. Acad. Sci. USA, 1993, 90:5873-7). FASTA search method is described by W. R. Pearson (“Rapid and Sensitive Sequence Comparison with FASTP and FASTA”, Methods in Enzymology, 1990 183:63- 98). ClustalW method is described by Thompson J. D., Higgins D. G. and Gibson T. J. (“CLUSTAL W: improving the sensitivity of progressive multiple sequence alignment through sequence weighting, position-specific gap penalties and weight matrix choice”, Nucleic Acids Res. 1994, 22:4673-4680).

Alternatively, the stringent conditions are exemplified by a condition under which DNA's are hybridized with each other at a salt concentration corresponding to an ordinary condition of washing in Southern hybridization, i.e., 60° C., 1×SSC, 0.1% SDS, preferably 0.1×SSC, 0.1% SDS. The duration of the washing procedure depends on the type of membrane used for blotting and, as a rule, is recommended by manufacturer. For example, recommended duration of washing for the Hybond™ N+ nylon membrane (Amersham) under stringent conditions is 15 minutes. Techniques for enhancing the expression of genes, especially techniques for increasing the number of protein molecules in a bacterial cell, include altering the expression regulatory sequence of a DNA coding for the protein, and increasing the copy number of the gene, but are not limited thereto.

Altering the expression regulatory sequence of a DNA can be achieved by placing the DNA coding for the protein of present invention under the control of a strong promoter. For example, lac promoter, trp promoter, trc promoter, PL promoter of lambda phage are all known as strong promoters. Alternatively, a promoter's activity can be enhanced by, for example, introducing a mutation into the promoter to increase the transcription level of a gene located downstream of the promoter. Furthermore, the mRNA translatability can be enhanced by introducing a mutation into a spacer between the ribosome binding site (RBS) and the start codon. For example, a 20-fold range in expression levels was found depending on the nature of the three nucleotides preceding the start codon (Gold et al., Annu. Rev. Microbiol., 35, 365-403, 1981; Hui et al., EMBO J., 3, 623-629, 1984).

Furthermore, to increase the transcription level of the gene, an enhancer may be newly introduced. Introduction of DNA containing either a gene or promoter into chromosomal DNA is described in, for example, Japanese Patent Application Laid-Open No. 1-215280 (1989).

Alternatively, the copy number of the gene may be increased by inserting the gene into a multi-copy vector to form a recombinant DNA, followed by introduction of the recombinant DNA into a microorganism. Preferably, multi-copy vectors are used. Examples of multi-copy vectors include, but are not limited to, pBR322, pUC19, pBluescript KS+, pACYC177, pACYC184, pAYC32, pMW119, pET22b or the like. The term “multi-copy vector” is used for vectors which result in a copy number of up to 15-30 copies per cell. Methods of transformation include any known methods in the art. For example, a method of treating recipient cells with calcium chloride so as to increase permeability of the cells to DNA has been reported for Escherichia coli K-12 (Mandel, M. and Higa, A., J. Mol. Biol., 53, 159 (1970)) and may be used in the present invention.

Enhancement of gene expression may also be achieved by introduction of multiple copies of the gene into a bacterial chromosome via, for example, homologous recombination, Mu integration or the like. For example, one act of Mu integration permits introduction into a bacterial chromosome of up to 3 copies of the gene.

The techniques described above using a strong promoter or enhancer can be combined with the techniques based on increasing the copy number or expression of the gene.

A preferable embodiment of the bacterium of the present invention can be obtained by enhancing the expression of the leuABCD operon of a bacterium which already has inactive AHASes. Alternatively, the preferable embodiment of the bacterium of the present invention can be obtained by inactivation of AHASes in a bacterium which inherently has enhanced expression of the leuABCD operon. Particularly, any known L-leucine producing strain can be used for imparting the ability to produce abnormal amino acids, such as norleucine and norvaline.

Examples of bacteria belonging to the genus Escherichia which have an activity to produce L-leucine include, but are not limited to, E. coli strains such as H-9068 (ATCC 21530), H-9070 (FERM BP-4704) and H-9072 (FERM BP-4706) resistant to 4-azaleucine or 5,5,5-trifluoroleucine (U.S. Pat. No. 5,744,331), E. coli strains in which feedback inhibition of isopropylmalate synthase by L-leucine is desensitized (European patent EP1067191), E. coli strain AJ11478 resistant to β-2-thienylalanine and β-hydroxyleucine (U.S. Pat. No. 5,763,231), E. coli strain 57 (VKPM B-7386, U.S. Pat. No. 6,124,121) and the like.

Methods for preparation of plasmid DNA, digestion and ligation of DNA, transformation, selection of an oligonucleotide as a primer and the like may be ordinary methods well known to one skilled in the art. These methods are described, for instance, in Sambrook, J., Fritsch, E. F., and Maniatis, T., “Molecular Cloning A Laboratory Manual, Second Edition”, Cold Spring Harbor Laboratory Press (1989).

2. Method of the Present Invention

The method of the present invention is a method for producing abnormal amino acids, which method includes the steps of cultivating the bacterium of the present invention in a culture medium which does not contain L-leucine, but does contain L-valine at a concentration which limits bacterial growth, so that the amino acid is produced and secreted into the medium, and collecting the amino acid from the medium. More specifically, the method of the present invention is a method for producing norleucine and/or norvaline, which method includes the steps of cultivating the bacterium of the present invention in a culture medium which does not contain L-leucine, but does contain L-valine at a concentration which limits bacterial growth, so that norleucine and/or norvaline is produced and secreted into the medium, and collecting norleucine and/or norvaline from the medium.

AHAS-deficient strains are not able to grow in the absence of valine and isoleucine in fermentation medium (valine and isoleucine auxotrophy). The fermentation medium should not contain L-leucine so as to avoid repression of the leucine operon (leuABCD operon). Lack of L-leucine necessary for cell growth is complemented by the presence of L-valine, which is partially deaminated to 2-ketoisovalerate—a precursor of L-leucine. On the other hand, a sufficient amount of added L-isoleucine is necessary in the fermentation medium to complement L-isoleucine auxotrophy.

The term “valine at concentrations limiting bacterial growth” means that the concentration of valine added into the fermentation medium is insufficient to provide the complete valine requirement for growing biomass in the absence of valine biosynthesis. The growth of the strain is therefore restricted by insufficient valine.

In the present invention, the cultivation, the collection and purification of an abnormal amino acid from the medium and the like may be performed in a manner similar to conventional fermentation methods wherein an amino acid is produced using a bacterium.

A medium used for culture may be either a synthetic medium or a natural medium, so long as the medium includes a carbon source and a nitrogen source and minerals and, if necessary, appropriate amounts of nutrients which the bacterium requires for growth. The carbon source may include various carbohydrates such as glucose and sucrose, and various organic acids. Depending on the mode of assimilation of the used microorganism, alcohol including ethanol and glycerol may be used. As the nitrogen source, various ammonium salts such as ammonia and ammonium sulfate, other nitrogen compounds such as amines, a natural nitrogen source such as peptone, soybean-hydrolysate, and digested fermentative microorganism may be used. As minerals, potassium monophosphate, magnesium sulfate, sodium chloride, ferrous sulfate, manganese sulfate, calcium chloride, and the like may be used. As vitamins, thiamine, yeast extract, and the like may be used.

The cultivation is performed preferably under aerobic conditions such as a shaking culture, and stirring culture with aeration, at a temperature of 20 to 40° C., preferably 30 to 38° C. The pH of the culture is usually between 5 and 9, preferably between 6.5 and 7.2. The pH of the culture can be adjusted with ammonia, calcium carbonate, various acids, various bases, and buffers. Usually, a 1 to 5-day cultivation leads to the accumulation of the target amino acid in the liquid medium.

After cultivation, solids such as cells can be removed from the liquid medium by centrifugation or membrane filtration, and then the amino acid can be collected and purified by ion-exchange, concentration, and crystallization methods.

### EXAMPLES

The present invention will be more concretely explained below with reference to the following non-limiting Examples. In the following Examples, norvaline and norleucine are in the L-form.

### Example 1

### Construction of the Strain Having Inactivated Acetolactate Synthases Genes

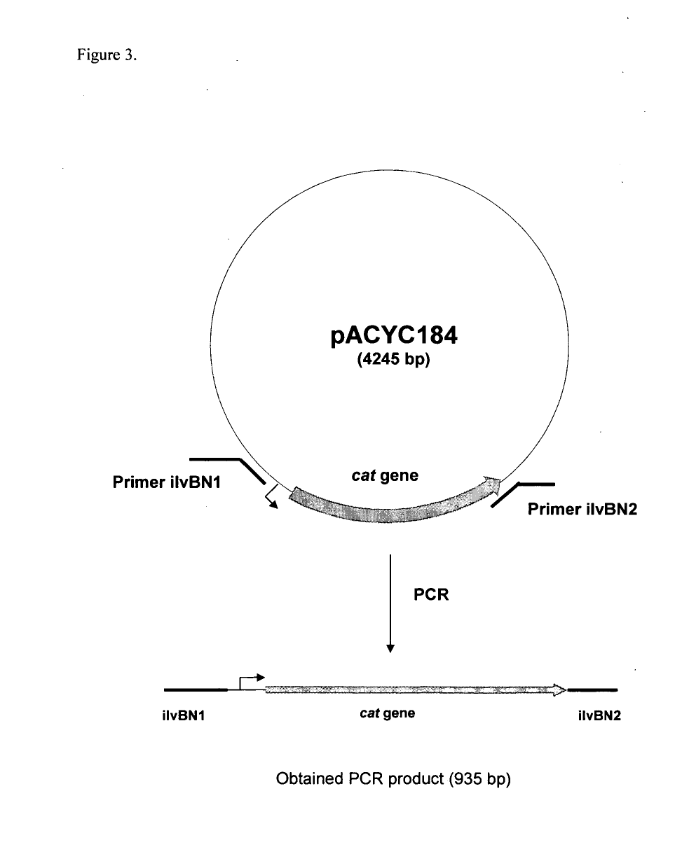

Unlike other wild-type E. coli strains, E. coli K-12 contains a polar frameshift mutation in the ilvG gene (Lawther, R. P. et al, Proc. Natl. Acad. Sci. USA 78:922-925, 1981). Thus, to obtain a strain having all AHASes inactivated from E. coli K-12, it is necessary to inactivate only two operons, ilvBN and ilvIH. See FIG. 3.

1. Deletion of the ilvBN Operon

Deletion of the ilvBN operon was constructed by means of the method firstly developed by Datsenko and Wanner (Proc. Natl. Acad. Sci. USA, 2000, 97(12), 6640-6645) called “Red-driven integration”. According to this procedure, the PCR primers ilvBN1 (SEQ ID NO: 1) and ilvBN2 (SEQ ID NO: 2), which are homologous to both regions adjacent to the ilvBN operon and the gene conferring chloramphenicol resistance in the template plasmid were constructed. The plasmid pACYC 184 (NBL Gene Sciences Ltd., UK) (GenBank/EMBL accession number X06403) was used as a template in PCR reaction. Conditions for PCR were the following: denaturation step for 3 min at 95° C.; profile for two first cycles: 1 min at 95° C., 30 sec at 34° C., 40 sec at 72° C.; profile for the last 30 cycles: 30 sec at 95° C., 30 sec at 50° C., 40 sec at 72° C.; final step: 5 min at 72° C.

The obtained 935 bp PCR product (SEQ ID NO: 3) was purified in an agarose gel and used for electroporation of the E. coli strain E. coli K-12 (VKPM B-7), containing the plasmid pKD46 with a temperature-sensitive replication. The plasmid pKD46 (Datsenko and Wanner, Proc. Natl. Acad. Sci. USA, 2000, 97:12:6640-45) includes a 2,154 nt (31088-33241) DNA fragment of phage λ (GenBank accession No. J02459), and contains genes of the λ Red homologous recombination system (γ, β, exo genes) under the control of arabinose-inducible P_{araB} promoter. The plasmid pKD46 is necessary for integration of the PCR product into the chromosome of strain E. coli K-12.

Electrocompetent cells were prepared as follows: an overnight culture of E. coli K-12 grown at 30° C. in LB medium supplemented with ampicillin (100 mg/l) was diluted 100 times with 5 ml of SOB medium (Sambrook et al, “Molecular Cloning A Laboratory Manual, Second Edition”, Cold Spring Harbor Laboratory Press (1989)) with ampicillin and L-arabinose (1 mM). The obtained culture was grown with aeration at 30° C. to an OD₆₀₀ of ≈0.6 and then made electrocompetent by concentrating 100-fold and washing three times with ice-cold deionized H₂O. Electroporation was performed using 70 μl of cells and ≈100 ng of PCR product. Following electroporation, cells were incubated with 1 ml of SOC medium (Sambrook et al, “Molecular Cloning A Laboratory Manual, Second Edition”, Cold Spring Harbor Laboratory Press (1989)) at 37° C. for 2.5 h and then plated onto L-agar and grown at 37° C. to select Cm^{R} recombinants. Then, to eliminate the pKD46 plasmid, 2 passages on L-agar with Cm at 42° C. were performed and the obtained colonies were tested for sensitivity to ampicillin.

2. Verification of the ilvBN Operon Deletion by PCR.

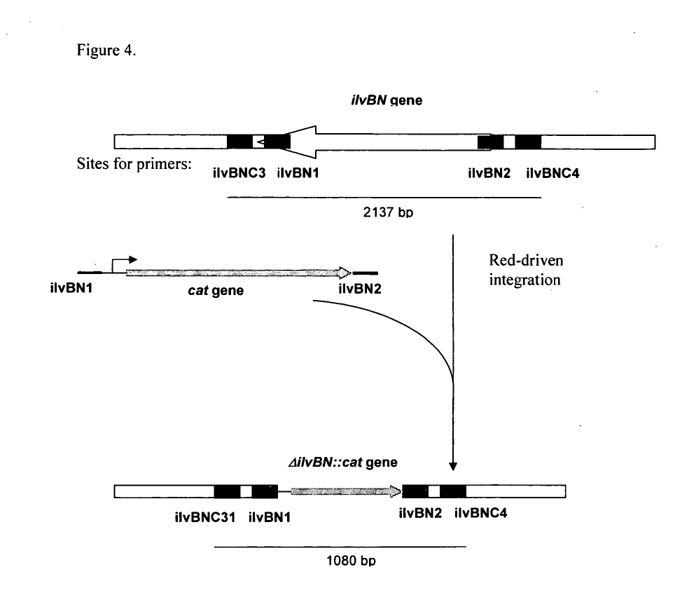

Mutants containing the deletion of ilvBN operon marked with Cm resistance gene were verified by PCR. Locus-specific primers ilvBNC3 (SEQ ID NO: 4) and ilvBNC4 (SEQ ID NO: 5) were used in PCR for the verification. Conditions for PCR verification were the following: denaturation step for 3 min at 94° C.; profile for the 30 cycles: 30 sec at 94° C., 30 sec at 53° C., 1 min at 72° C.; final step: 7 min at 72° C. PCR product, obtained in the reaction with the chromosomal DNA from parental ilvBN⁺ strain K-12 as a template, was 2137 nt in length (FIG. 4). PCR product, obtained in the reaction with the chromosomal DNA from the ilvBN deletion mutant named B-7 ΔilvBN: :cat strain as a template, was 1080 nt in length (FIG. 4, SEQ ID NO: 6).

3. Deletion of the ilvIH operon

Deletion of the ilvIH operon was conducted using the same approach as the deletion of the ilvBN operon described in Section 1. According to this procedure, the PCR primers ilvIHI1 (SEQ ID NO: 7) and ilvIHI2 (SEQ ID NO: 8), which are homologous to both regions adjacent to the ilvIH operon and the gene conferring kanamycine resistance in the template plasmid were constructed. The plasmid pACYC 177 (NBL Gene Sciences Ltd., UK) (GenBank/EMBL accession number X06402) was used as a template in PCR reaction. Conditions for PCR were the following: denaturation step for 3 min at 95° C.; profile for two first cycles: 1 min at 95° C., 30 sec at 34° C., 40 sec at 72° C.; profile for the last 30 cycles: 30 sec at 95° C., 30 sec at 50° C., 40 sec at 72 ° C.; final step: 5 min at 72° C.

The obtained 1370 bp PCR product (SEQ ID NO: 9) was purified in agarose gel and used for electroporation of the E. coli strain B-7ΔilvBN::cat containing the temperature-sensitive plasmid pKD46, which harbors genes of the λ Red homologous recombination system (γ, β, exo genes) under the control of the arabinose-inducible P_{araB} promoter. Kanamycine-resistant recombinants were selected after electroporation and verified by PCR with the locus-specific primers ilvIHC3 (SEQ ID NO: 10) and ilvIHC4 (SEQ ID NO: 11). Conditions for PCR verification were the following: denaturation step for 3 min at 94° C.; profile for the 30 cycles: 30 sec at 94° C., 30 sec at 53° C., 1 min 20 sec at 72° C.; final step: 7 min at 72° C. The PCR product, obtained in the reaction with the chromosomal DNA from parental IlvIH⁺ strain B-7 ΔilvBN::cat as a template, was 2486 nt in length. The PCR product, obtained in the reaction with the chromosomal DNA from mutant B-7 ΔilvBN::cat ΔilvIH::KmR strain as a template, was 1475 nt in length (SEQ ID NO: 12). As a result the strain B-7 ΔilvBN::cat ΔilvIH::KmR was obtained and named as NS1118. Strain NS1118 exhibits isoleucine and valine auxotrophy.

### Example 2

### Production and Secretion of Norleucine and Norvaline by the E. coli Strain NS1118

E. coli strains K-12 and NS1118 were grown for 18 hours at 37° C. on L-agar plates. Then cells from about 0.5 cm² of the plate surface were introduced into the fermentation medium (2 ml) and cultivated for 72 hours at 32° C. Accumulated amounts of norvaline and norleucine were measured by HPLC. For the detailed description of the measurement, see Example 6. The results are presented in Table 1.

The composition of the fermentation medium (g/l):

| | |
|---|---|
| Glucose | 60.0 |
| (NH₄)₂SO₄ | 18.0 |
| KH₂PO₄ | 2.0 |
| MgSO₄ × 7H₂O | 1.0 |
| CaCO₃ | 25.0 |
| Thiamin | 0.02 |

TABLE 1

| Strain | Additions* | Norvaline, g/l | Norleucine, g/l |
|---|---|---|---|
| K-12 | — | <0.1 | <0.1 |
| NS1118 | Ile, Val | 0.9 | 1.9 |
| NS1118 | Ile, Val, Leu | <0.1 | <0.1 |
| NS1118 | Ile, Val** | <0.1 | <0.1 |

*Ile - 100 μg/ml, Val - 100 μg/ml, Leu - 100 μg/ml,
**Val - 500 μg/ml

As it is seen from Table 1, the E. coli strain NS1118, in which all the AHASes were inactive, produced and secreted norvaline and norleucine unlike the wild-type parent strain K-12. Addition of leucine prevented production and secretion of abnormal amino acids, presumably because of isopropylmalate synthase inhibition.

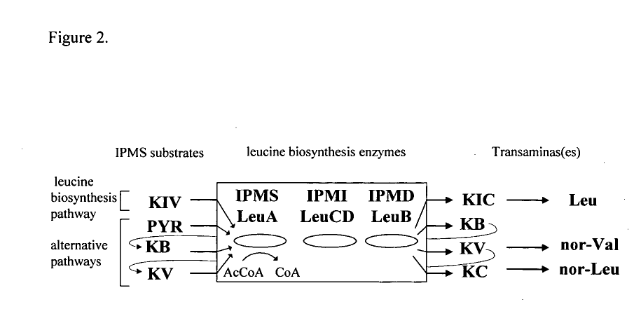

Addition of valine at growth-limiting concentrations is necessary for production of abnormal amino acids. In this scenario, acetolactate deficiency results in 2-ketobutyrate pool elevation. Limiting the amount of valine resulted in a 2-ketoisovalerate deficiency, which, in turn, provoked a leucine synthesis decrease. Limiting the amount of leucine derepressed the leucine operon. 2-ketobutyrate was utilized by isopropylmalate synthase as an alternative substrate (instead of 2-ketoisovalerate) and further metabolized into nor-valine and nor-leucine by other enzymes, encoded by the leucine operon (FIG. 2). Increasing the concentration of valine in the initial fermentation medium also prevented norleucine and norvaline synthesis by increasing the ketoisovalerate/2-ketobutyrate ratio.

Therefore, specific growth conditions are necessary for such abnormal amino acids synthesis: the absence of leucine and the presence of valine in the fermentation medium. Also, valine must be at a concentration which limits bacterial growth.

### Example 3

### Cloning of AHASIII

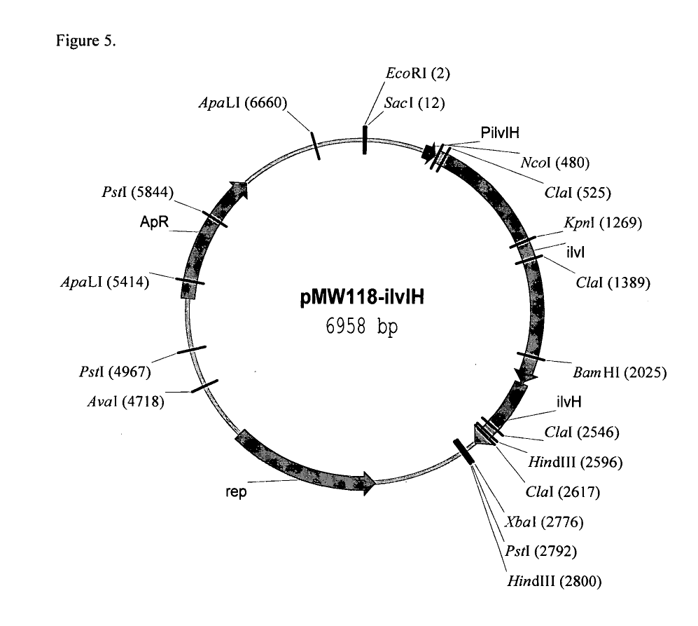

The ilvIH operon was cloned on pMW118 vector as a 2764 bp PCR product. The chromosome DNA of E. coli strain MG1655 (CGSC6300) was used as a template in the PCR reaction. Synthetic oligonucleotides ilvIHL58 (SEQ ID NO: 13) and ilvIHR60 (SEQ ID NO: 14) were used as primers. Primer ilvIHL58 contains the SacI-restriction site introduced in the 5′-end thereof and primer ilvIHR60 contains the XbaI-restriction site introduced in the 5′-end thereof. Conditions for PCR were the following: denaturation step for 3 min at 94° C.; profile for 30 cycles: 30 sec at 94° C., 30 sec at 57° C., 2 min at 72° C.; final step: 7 min at 72° C. Obtained 2778 bp PCR product was purified in agarose gel, digested with SacI and XbaI, and cloned into pMW118 vector previously digested with the same restrictases. The strain NS1118 was used as a recipient for cloning. The resulted plasmid pMW118-ilvIH (FIG. 5) complemented AHAS⁻ phenotype of the NS1118 strain.

### Example 4

### Cloning of Leucine Operon

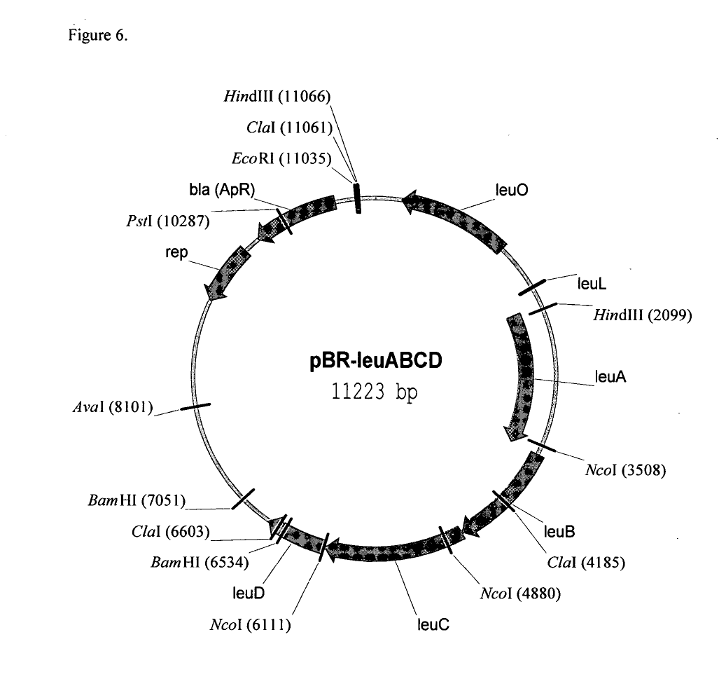

The leuABCD operon was cloned on the pBR322 vector by the shotgun method. For this purpose the chromosome of MG1655 strain was digested with MfeI, blunted with Klenow fragment and ligated with plasmid pBR322 linearized with EcoRV. The E. coli strain leu::Tn10 (VKPM B-6038) was used as a recipient for cloning. As a result the plasmid pBR-leuABCD containing 6.9 kb chromosome fragment, which harbors the leuABCD operon was obtained (FIG. 6).

### Example 5

### Production and Secretion of Norleucine and Norvaline by Derivatives of the Strain NS1118

E. coli strain NS1118 and its derivatives were grown for 18 hours at 37° C. on L-agar plates containing ampicillin (100 μg/ml) in the case of plasmid strains. Then cells from about 0.5 cm² of the plate surface were introduced into a fermentation medium (2 ml, Example 2) and cultivated for 72 hours at 32° C. Accumulated amounts of norvaline and norleucine were measured by HPLC. For the detailed description of the measurement see Example 6. The results are presented in Table 2.

TABLE 2

| Strain | Additions* | Norvaline, g/l | Norleucine, g/l |
|---|---|---|---|
| NS1118 | Ile, Val | 0.9 | 1.9 |
| NS1118/pMW118-ilvIH | — | <0.01 | <0.01 |
| NS1118/pBR-leuABCD | Ile, Val | 0.78 | 3.9 |

*Ile- 100 μg/ml, Val- 100 μg/ml

As it is seen from Table 2, restoring AHAS activity by introducing the plasmid pMW118-ilvIH which harbors the ilvIH genes (encoding AHAS III), blocked norvaline and norleucine production and secretion by the AHAS-deficient strain NS1118. Amplification of the wild-type leucine operon as a part of plasmid pBR-leuABCD increased norleucine accumulation in the medium.

### Example 6

### Chromatographic Conditions for Analysis of Norvaline and Norleucine in the Culture Medium after Fermentation

The Waters AccQ-Tag® Method was used for the analysis. The AccQ-Tag Method is a precolumn derivatization technique for amino acid determination. The HPLC system Alliance 2690 gradient system (Waters Corp.) equipped with 1100 series fluorescence detector (Agilent Technologies) and connected to computer loaded with “ChemStation A.08.04” chromatography software (Agilent Technologies) was used. The detector settings were the following: excitation wavelength—250 nm, emission wavelength—395 nm. 5 μl sample injection loop was used for all runs. Waters AccQ-Tag Amino Acid Analysis Column equipped with a guard column separated the amino acid derivatives produced by the Accq-fluor derivatization reaction. The column was thermostated in the column oven at 37° C. The mobile-phase flow-rate was set at 1 ml/min. The mobile phases consisted of: (B) aqueous buffer “Waters AccQ-Tag Eluent A”, (C) HPLC-grade acetonitrile and (D) Mili-Q water.

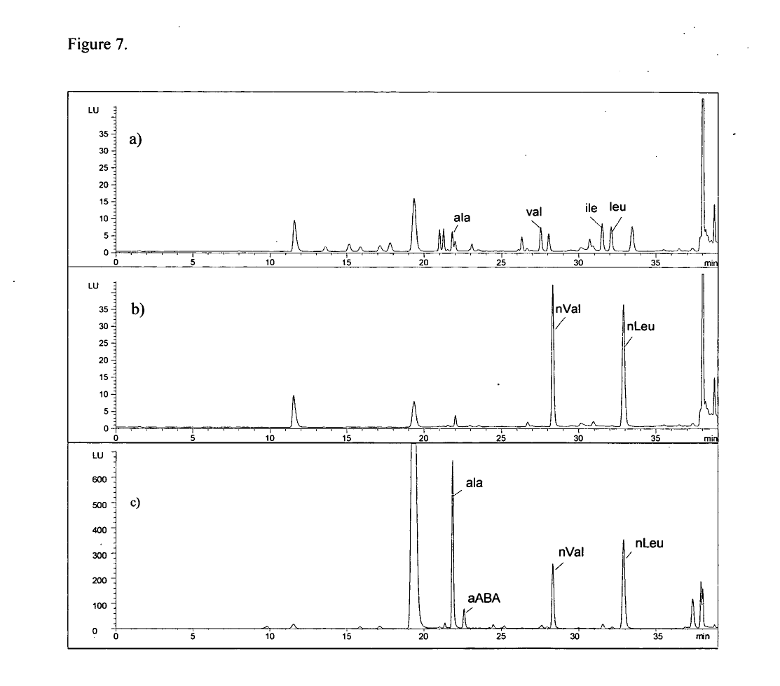

The gradient program for chromatographic amino acid analysis is shown in Table 3. Chromatographic amino acid analysis profiles are presented on FIG. 7.

TABLE 3

| | Time when the | Eluent | Eluent | Eluent, | |
|---|---|---|---|---|---|
| Step | step starts, min | B, % | C, % | D % | Curve |
| 1 | 0 | 100.0 | 0.0 | 0 | 0 |
| 2 | 0.50 | 99.0 | 1.0 | 0 | 11 |
| 3 | 18.00 | 95.0 | 5.0 | 0 | 6 |
| 4 | 19.00 | 91.0 | 9.0 | 0 | 6 |
| 5 | 29.50 | 83.0 | 17.0 | 0 | 6 |
| 6 | 33.00 | 73.0 | 22.0 | 5 | 11 |
| 7 | 36.00 | 0.0 | 60.0 | 40 | 11 |
| 8 | 39.00 | 100.0 | 0.0 | 0 | 11 |
| 9 | 48.00 | 100.0 | 0.0 | 0 | 11 |

Curve 6 is a linear segment; curve 11 is a step function.

While the invention has been described in detail with reference to preferred embodiments thereof, it will be apparent to one skilled in the art that various changes can be made, and equivalents employed, without departing from the scope of the invention. Each of the aforementioned documents, including the foreign priority document, RU 2004127011, is incorporated by reference herein in its entirety.

What is claimed is:

1. An abnormal amino acid-producing bacterium of the Enterobacteriaceae family, wherein the bacterium has been modified to inactivate all acetohydroxy acid synthases (AHASes).

2. The bacterium according to claim 1, wherein said abnormal amino acid is norleucine and/or norvaline.

3. The bacterium according to claim 1, wherein the bacterium belongs to the genus Escherichia.

4. The bacterium according to claim 3, wherein said acetohydroxy acid synthases comprise AHAS I encoded by the ilvBN genes, AHAS II encoded by the ilvGM genes, and AHAS III encoded by the ilvIH genes.

5. The bacterium according to claim 4, wherein said bacterium is modified to have enhanced expression of the leuABCD operon.

6. A method for producing abnormal amino acid, which method comprises:
cultivating the bacterium according to claim 1 in a medium which does not contain L-leucine, but contains L-valine at a concentration which limits bacterial growth, so that said abnormal amino acid is produced and secreted into the medium, and
collecting the abnormal amino acid from the medium.

7. The method according to claim 6, wherein said abnormal amino acid is norleucine and/or norvaline.

8. A method for producing abnormal amino acid, which method comprises:
cultivating the bacterium according to claim 2 in a medium which does not contain L-leucine, but contains L-valine at a concentration which limits bacterial growth, so that said abnormal amino acid is produced and secreted into the medium, and
collecting the abnormal amino acid from the medium.

9. The method according to claim 8, wherein said abnormal amino acid is norleucine and/or norvaline.

10. A method for producing abnormal amino acid, which method comprises:
cultivating the bacterium according to claim 3 in a medium which does not contain L-leucine, but contains L-valine at a concentration which limits bacterial growth, so that said abnormal amino acid is produced and secreted into the medium, and
collecting the abnormal amino acid from the medium.

11. The method according to claim 10, wherein said abnormal amino acid is norleucine and/or norvaline.

12. A method for producing abnormal amino acid, which method comprises:
cultivating the bacterium according to claim 4 in a medium which does not contain L-leucine, but contains L-valine at a concentration which limits bacterial growth, so that said abnormal amino acid is produced and secreted into the medium, and
collecting the abnormal amino acid from the medium.

13. The method according to claim 12, wherein said abnormal amino acid is norleucine and/or norvaline.

14. A method for producing abnormal amino acid, which method comprises:
cultivating the bacterium according to claim 5 in a medium which does not contain L-leucine, but contains L-valine at a concentration which limits bacterial growth, so that said abnormal amino acid is produced and secreted into the medium, and
collecting the abnormal amino acid from the medium.

15. The method according to claim 14, wherein said abnormal amino acid is norleucine and/or norvaline.
